(19) United States
(12) Patent Application Publication
(10) Pub. No.: US 2026/0053413 A1
(43) Pub. Date: Feb. 26, 2026

(54) BACKGROUND MAGNETIC FIELD COMPENSATION IN MAGNETIC FIELD DETECTION SYSTEMS

(71) Applicant: FieldLine Inc., Boulder, CO (US)

(72) Inventors: Svenja Knappe, Boulder, CO (US); Orang Alem, Erie, CO (US); Kenneth J. Hughes, Lafayette, CO (US)

(21) Appl. No.: 19/308,630

(22) Filed: Aug. 25, 2025

Related U.S. Application Data
(60) Provisional application No. 63/686,248, filed on Aug. 23, 2024.

(51) Int. Cl. A61B 5/245; G01R 33/032; G01R 33/09; G01R 33/26

(52) U.S. Cl. CPC A61B 5/245; G01R 33/032; G01R 33/09; G01R 33/26; A61B 2562/0223

(57) ABSTRACT

Various embodiments disclosed herein comprise a system. The system comprises a coil, a magnetic field sensor, and a controller. The coil generates a spatially and temporally varying magnetic field. The magnetic field sensor measures the magnetic field and a background magnetic field. The magnetic field sensor provides a magnetic field measurement and a background magnetic field measurement to the controller. The controller determines the position and orientation of the magnetic field sensor based on the magnetic field measurement and the background magnetic field measurement. The controller transfers control signaling to modify the magnetic field based on the position and orientation of the magnetic field sensor to null the background magnetic field. The coil receives the control signaling and modifies the spatially and temporally varying magnetic field based on the control signaling to null the background magnetic field.

### CROSS-REFERENCE TO RELATED APPLICATIONS

This U.S. Patent application claims the benefit of and priority to U.S. Provisional Patent Application 63/686,248 titled, “DYNAMIC GLOBAL MAGNETIC FIELD CONTROL APPARATUS FOR MOBILE MAGNETIC MEASUREMENT” which was filed on Aug. 23, 2024. U.S. Provisional Patent Application 63/686,248 is hereby incorporated by reference in its entirety into this U.S. Patent Application.

### TECHNICAL FIELD

Various embodiments of the present technology relate to nulling background magnetic fields, and more specifically, utilizing the sensor output to dynamically control compensation fields to null background magnetic fields.

### BACKGROUND

Magnetic field detection systems detect and characterize magnetic fields generated by a magnetic field source. The magnetic field detection systems measure metrics like the strength and direction of the magnetic fields to characterize the sensed fields. Biomagnetic Fields (MXG) systems are a type of magnetic field detection system that measures magnetic fields generated by neural activity within the human body to map bodily functions. MXG systems image neural or other biological electromagnetic activity by detecting the magnetic component of electromagnetic fields produced by the body using an array of magnetic field sensors placed on or near the subject. The MXG systems compute the location of the biological electromagnetic activity relative to the location of the magnetic field sensors in a process referred to as source localization. The data from the magnetic field sensors along with the locations of the magnetic field sensors are used to calculate the location of the biological electromagnetic activity to form an MXG image of the biological electric activity. The MXG image provides information about the electrophysiology of the bodily function (e.g., neural currents in the human brain). Exemplary MXG types include Magnetoencephalography (MEG), Magnetocardiography (MCG), Magnetogastrography (MGG), Magnetomyography (MMG), and Magnetoneurography (MNG). Exemplary magnetic field sensors used in MXG systems include magnetometers, atomic magnetometers, Optically Pumped Magnetometers (OPMs), Superconducting Quantum Interference Devices (SQUIDs), Nitrogen Vacancy Centers (NVs), Magnetoresistive (MR) sensors, and the like.

Some MXG systems have sensors that move independently and conform to the size and shape of the body. These MXG systems are referred to as on-body or conformal MXG. Conformal MXG systems utilize wearable devices like headgear, vests, and sleeves to conform the magnetic field sensors to the body. To effectively relate the measured magnetic fields to neural activity, the location and orientation information for the magnetic field sensors is determined for every subject and every time the sensors are placed on the body to allow for accurate source localization of the electrical activity in the body. One of the advantages of conformal MXG systems is that the subject is able to move during magnetic recordings.

Since the environment around the subject often produces magnetic fields that are large compared to the magnetic fields emitted by the body, artifacts from these environmental fields are suppressed to generate MXG images. The artifacts from the environmental fields often have a static and time dependent component. When the subject is moving during recording, the artifacts from the static environmental fields also cause the sensor readings to vary with time. The environmental fields are mitigated with passive and active shields. Passive shields comprise materials with high magnetic permeability like permalloys, mu-metal, and the like. Active shields comprise coils and/or magnets that produce magnetic fields that counteract the environmental fields. In order to make dynamic adjustments to cancel the time-varying magnetic fields and gradients, the environmental magnetic field is measured by static reference sensors near the MXG apparatus that mounts the magnetic field sensors. While static reference sensors may be used to control the active shielding, they are not co-located with the MXG apparatus. This results in the nulling field produced by the coils to be tailored for the shape and magnitude of the background magnetic at the location of the static reference sensors, not the location of the MXG apparatus. Unfortunately, conventional MXG systems do not effectively or efficiently mitigate the environmental magnetic fields.

### OVERVIEW

This Overview is provided to introduce a selection of concepts in a simplified form that are further described below in the Technical Description. This summary is not intended to identify key features or essential features of the claimed subject matter, nor is it intended to be used as an aid in determining the scope of the claimed subject matter.

Various embodiments of the present technology relate to solutions for environmental magnetic field mitigation. Some embodiments comprise a system. The system comprises a coil, a magnetic field sensor, and a controller. The coil generates a spatially and temporally varying magnetic field. The magnetic field sensor measures the magnetic field and a background magnetic field. The magnetic field sensor provides a magnetic field measurement and a background magnetic field measurement to the controller. The controller determines the position and orientation of the magnetic field sensor based on the magnetic field measurement. The controller transfers control signaling to modify the magnetic field based on the position and orientation of the magnetic field sensor and the background magnetic field measurement to null the background magnetic field. The coil receives the control signaling and modifies the spatially and temporally varying magnetic field based on the control signaling to null the background magnetic field.

Some embodiments comprise a method. The method comprises generating, by a coil, a spatially and temporally varying magnetic field. The method further comprises measuring, by a magnetic field sensor, the magnetic field and a background magnetic field. The method further comprises providing, by a magnetic field sensor, a magnetic field measurement and a background magnetic field measurement to a controller. The method further comprises determining, by the controller, a position and orientation of the magnetic field sensor based on the magnetic field measurement. The method further comprises transferring, by the controller, control signaling to modify the magnetic field based on the position and orientation of the magnetic field sensor and the background magnetic field measurement to null the background magnetic field. The method further comprises receiving, by the coil, the control signaling. The method further comprises modifying, by the coil, the spatially and temporally varying magnetic field based on the control signaling to null the background magnetic field.

Some embodiments comprise one or more non-transitory computer readable storage media. The computer readable storage media store instructions that, when executed by a computing system, direct the computing system to perform operations. The operations comprise controlling a coil to generate a spatially and temporally varying magnetic field. The operations further comprise directing a magnetic field sensor to measure the magnetic field and a background magnetic field. The operations further comprise obtaining a magnetic field measurement and a background magnetic field measurement. The operations further comprise determining the location and orientation of the magnetic field sensor based on the magnetic field measurement. The operations further comprise controlling the coil to modify the magnetic field based on the location and orientation of the magnetic field sensor and the background magnetic field measurement to null the background magnetic field.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an example of a magnetic field detection system.

FIG. 2 illustrates an example operation of the magnetic field detection system.

FIG. 3 illustrates an example of a Magnetoencephalography (MEG) system.

FIG. 4 illustrates an example of the MEG system.

FIG. 5 illustrates an example operation of the MEG system.

FIG. 6 illustrates an example operation of the MEG system.

FIG. 7 illustrates an example operation of the MEG system.

FIG. 8 illustrates an example computing system.

The drawings have not necessarily been drawn to scale. Similarly, some components or operations may not be separated into different blocks or combined into a single block for the purposes of discussion of some of the embodiments of the present technology. Moreover, while the technology is amendable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### TECHNICAL DESCRIPTION

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

Magnetoencephalography (MEG) systems measure the magnetic component of electromagnetic fields generated by neural activity in the human brain. The measured magnetic fields are plotted to map the neural activity in the brain. MEG systems rely on MEG sensors like magnetic field sensors, magnetometers, atomic magnetometers, Optically Pumped Magnetometers (OPMs), Superconducting Quantum Interference Devices (SQUIDs), Nitrogen Vacancy Centers (NVs), Magnetoresistive (MR) sensors, and the like to measure the magnetic fields. The MEG systems utilize wearable headgear like rigid helmets or flexible caps to mount the magnetic field sensors. The MEG sensors record the magnetic fields produced by brain activity. The MEG sensors may also detect background magnetic fields. Often, the background magnetic fields are much larger than the brain fields may interfere with the operation of the MEG system. Arrays of reference magnetic field sensors are used to spatially distinguish between the signals from the background fields and the brain. The signals from the background fields generally have a stationary and a time-varying component. MEG systems employ active and passive shielding to block, minimize, or otherwise reduce the effects of the background magnetic fields. Passive shielding typically comprises mu-metal magnetic shielding or another material with high magnetic permeability. For example, a room may be plated with mu-metal panels to create a magnetically shielded room. While passive shielding blocks a portion of the background magnetic field, a portion of the background magnetic fields are able to pass through the passive shielding in the nanotesla range. This is still larger than most magnetic fields produced by the body which may degrade the quality of the measurements performed by the MEG sensors.

Active shields are typically used to augment passive shields. Active shields comprise one or more coils and/or magnets positioned on or near the passive shield that produce compensation magnetic fields. Directionally opposed magnetic fields of equal magnitude will cancel each other out resulting in a volume free of the magnetic fields (e.g., similar to destructive interference). Active shields apply opposing fields to cancel the remaining background magnetic field at locations of interest. Since spatial variations of the environmental magnetic fields exist, sets of coils are used to cancel the homogeneous components in all directions and the gradients over the volume of the magnetic sensor array. If the background magnetic field has time-varying components, the active shield adjusts the magnitude/direction of the compensation magnetic field to compensate for the changing field distribution. To achieve this, conventional MEG systems use reference magnetic field sensors to detect and report the changing strength and direction of the background magnetic field to a controller. The controller then provides current to the active shield to modify the compensation field based on the measurements to form a control loop. To simplify the operation, the reference sensors are stationary with known locations and orientations with respect to the active shield. This allows for decomposition of the magnetic fields into components to optimize the current distribution in the active shield coils. The MEG sensors are typically not used to control the compensating field because their locations and orientation are not always constant. While this allows for easy decomposition, the background distribution is not measured at the location where the field needs to be compensated as the reference sensors are not co-located with the MEG sensors. Since the background magnetic field is not measured at the location of the MEG sensors, the resulting compensation field produced based on the background field measurements is not tailored to counter the effects of the background field at the location/orientation of the MEG sensors. Unfortunately, this reduces the effectiveness of the compensation field and degrades the overall user experience.

To overcome the above-described problems in conventional MEG systems, various embodiments of the technology described herein relate to utilizing the MEG sensor output to dynamically control compensation fields to null background magnetic fields. A MEG controller drives MEG sensors to measure a background magnetic field, a compensation magnetic field, and typically a target magnetic field. The MEG controller extracts the location and orientation of the MEG sensors from the compensation magnetic field measurements. The MEG controller drives compensation coils to generate a compensation magnetic field based on the background magnetic field measurements and the location/orientation of the MEG sensors. As the background magnetic field (e.g., due to environmental changes) and the location/orientation of the MEG sensors change (e.g., due to head movement by the user), the MEG sensors report updated measurements of the background magnetic field and the compensation magnetic field. The MEG controller determines updated location/orientations of the MEG sensors and adjusts the compensation magnetic field based on the updated locations/orientations and updated background field measurements forming a control loop. Advantageously, the MEG system effectively and efficiently utilizes MEG sensor outputs to drive the operation of compensation coils. Moreover, this results in nulling the background magnetic field at the location of the MEG sensors which improves the effectiveness of the compensation field and enhances the overall user experience.

The MEG controller may drive the compensation coils to apply time-varying magnetic fields and magnetic field gradients to dynamically measure the MEG sensor locations. This may be achieved by applying sinusoidal field modulations. If the modulation is in a frequency band of interest to MEG sensor, the localization can be interleaved with the MEG measurements. While this is often not preferable, it yields a stable sensor response since the fields can be applied at a frequency where the MEG sensor response is reproducible. If the MEG sensor locations are determined simultaneously with the measurement of the target magnetic field, the field modulations may be applied outside the frequency band of interest to the MEG sensors. Some MEG sensors like OPMs have bandwidths similar to the frequency band of interest. In that case, when the field modulation is applied outside frequency band of interest, the response experiences a roll-off. The roll-off is difficult to control over long periods of time and in changing environments. Additional frequency tones may be applied to the MEG sensors (e.g., by onboard bias coils) to dynamically measure the roll-off and compensate for it. The dynamically measured sensor locations and directions are then used to determine a dynamic set of weights, which in turn determines the relative distribution of feedback to all the compensation coils. This allows decomposing the signals from the MEG sensors into those due to the movement of the subject and those due to time-varying environmental fields. This information may then be used to dynamically compensate for spatially and temporally varying environmental fields at the location of the MEG sensors while the sensors are moving. Now referring to the Figures.

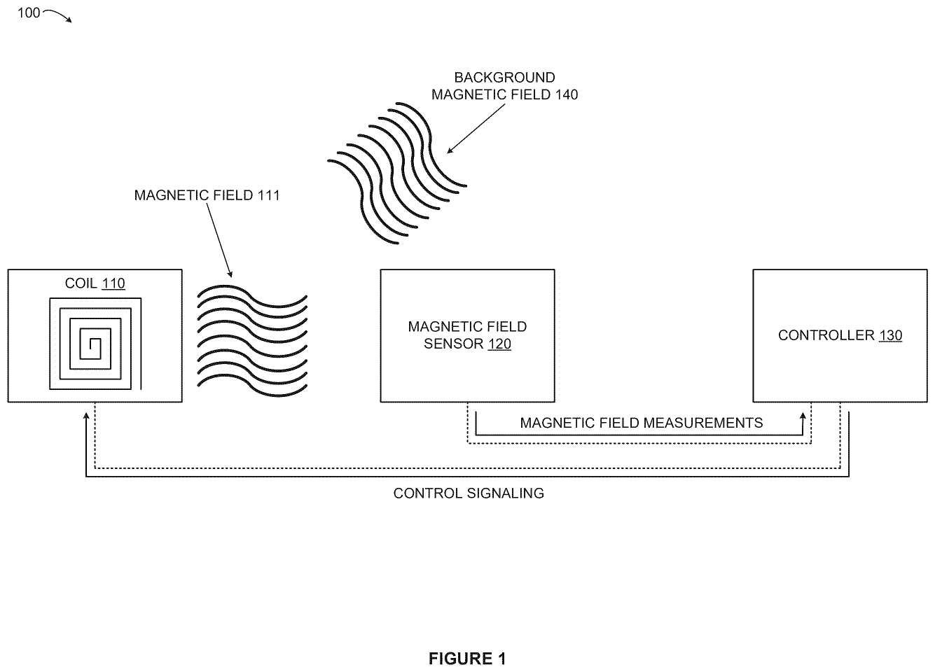

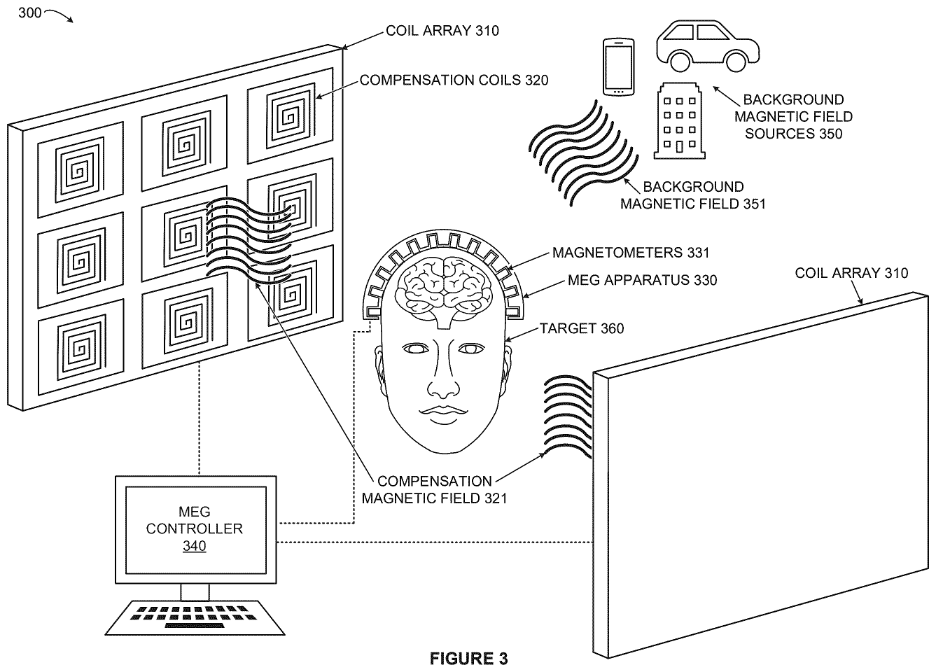

FIG. 1 illustrates an example of magnetic field detection system 100 to mitigate background magnetic fields and background magnetic field 140. Magnetic field detection system 100 performs operations like detecting target magnetic fields, nulling background magnetic fields at a desired location, and relating the target magnetic fields to information of interest. For example, magnetic field detection system 100 may comprise a Biomagnetic Fields (MXG) system. Magnetic field detection system 100 comprises an example of MEG system 300 illustrated in FIGS. 3-7, however MEG system 300 may differ. In some examples, magnetic field detection system 100 comprises coil 110, magnetic field 111, magnetic field sensor 120, and controller 130. In other examples, magnetic field detection system 100 may include additional or different components than illustrated in FIG. 1. Generally, magnetic field detection system 100 measures a target magnetic field generated by a target, however the target and the target magnetic field are omitted for clarity. For example, the target may comprise any biological magnetic field source like the human head, torso, abdomen, and the like, or may comprise a non-biological magnetic field source.

Various examples of system configuration and operation are described herein. In some examples, controller 130 generates and transfers control signaling to coil 110. The control signaling drives coil 110 to generate magnetic field 111. Magnetic field 111 generated by coil 110 is a spatially and temporally varying magnetic field used to locate magnetic field sensor 120 and to mitigate, null, or otherwise counter the effects of background magnetic field 140. Background magnetic field 140 comprises an example of magnetic interference that degrades the ability of magnetic field sensor 120 to accurately measure magnetic fields of interest. Background magnetic field 140 may be generated by the operation of electronic devices and the movement of ferrous objects like cars, doors, elevators, and the like. In some examples, background magnetic field 140 may be representative of the Earth's magnetic field.

Magnetic field sensor 120 measures magnetic field 111 and background magnetic field 140. The position and orientation or magnetic field sensor 120 may change during the recording of magnetic field 111 and background magnetic field 140. For example, magnetic field sensor 120 may attach to a wearable apparatus (e.g., an MXG device) and measure the magnitude and direction of magnetic field 111 as well as the magnitude and direction of background magnetic field 140. Magnetic field sensor 120 generates and transfers magnetic field measurements that characterize magnetic field 111 and background magnetic field 140 to controller 130. Controller 130 determines the location and orientation of magnetic field sensor 120 based on the measurement of magnetic field 111. For example, controller 130 may drive coil 110 to apply homogenous magnetic fields and/or gradient magnetic fields in magnetic field 111. Controller 130 may then determine the location and/or of magnetic field sensor 120 by extracting information from the field components originating from coil 110. Controller 130 generates control signaling to modify magnetic field 111 based on the measurement of background magnetic field 140 and the location and orientation of magnetic field sensor 120. Controller 130 transfers the control signaling to coil 110. For example, controller 130 may apply a modulation pattern to the current supplied to coil 110 to drive coil 110 to modify magnetic field 111 based on the modulation pattern. Coil 110 modifies magnetic field 111 based on the control signaling to null background magnetic field 140 at the location and orientation of magnetic field sensor 120. For example, coil 110 may modify magnetic field 111 to be of equal magnitude and directionally inverted to background magnetic field 140 in a volume that encompasses magnetic field sensor 120. This reduces magnetic field noise induced by spatially and temporally varying background magnetic field 140 as magnetic field sensor 120 moves through and/or remains stationary in background magnetic field 140.

In should be appreciated the location and orientation of magnetic field sensor 120 may change over time. For example, magnetic field sensor 120 may be part of an array attached to a wearable device. The user wearing the device may move causing the location and orientation of magnetic field sensor 120 to change. As such, magnetic field sensor 120 may may repeatedly (e.g., continuously, periodically, semi-periodically, etc.) measure magnetic field 111 and background magnetic field 140. Controller 130 may then determine an updated location and orientation of magnetic field sensor 120, and generate control signaling to readjust magnetic field 111 to account for changes to the location/orientation of magnetic field sensor 120 as well as changes (e.g., strength, direction, etc.) to background magnetic field 140.

Advantageously, magnetic field detection system 100 effectively and efficiently utilizes the output of a sensor with a variable location/orientation to drive the operation of a compensation coil. Moreover, magnetic field detection system 100 nulls the background magnetic field at the location of the sensor which improves the effectiveness of the compensation field produced by the coil. This enhances the overall user experience.

Coil 110 comprises one or more loops of metallic wiring that generate an electromagnetic field in response to receiving electric current. Coil 110 may comprise single or multiple loops of any shape and size. Coil 110 may comprise sets of separated coils with differing loops of varying shapes, sizes, and orientations. The orientations and spatial configuration of coil 110 may vary. Although illustrated as comprising a single coil, in other examples, magnetic field detection system 100 may comprise additional coils. For example, coil 110 may comprise one coil of a coil array controlled by controller 130. Coil 110 may be mounted on a surface like a wall, the floor, the ceiling, a mobile mount, and the like. Coil 110 and magnetic field sensor 120 are typically positioned within a passive shield environment like a magnetically shielded room to partially mitigate background magnetic field 140. For example, the magnetically shielded room may comprise a mu-metal enclosure. Coil 110 may comprise one or more on-board processing elements. For example, coil 110 may comprise a local controller that interfaces with controller 130 to modify magnetic field 111 based on the control signaling. The local controller may receive the control signaling from controller 130, translate the control signaling into a modulation pattern, and modulate the current supplied to coil 110 based on the modulation pattern to apply the desired modifications to magnetic field 111 via coil 110.

Magnetic field sensor 120 comprises a device with capabilities to sense magnetic fields generated by a magnetic field source in a target, magnetic field 111 generated by coil 110, and background magnetic field 140. Magnetic field sensor 120 may comprise a magnetometer, atomic magnetometer, Optically Pumped Magnetometer (OPM), gradiometer, Nitrogen Vacancy Centers (NV), high-temperature Superconducting Quantum Interference Device (SQUID), a Magnetoresistive (MR) sensor, and the like. Magnetic field sensor 120 generates signals that characterize the strength and/or direction of the detected magnetic fields. Controller 130 is representative of one or more computing devices to control the operation of coil 110 and magnetic field sensor 120. Exemplary controller types include Proportional-Integral-Derivative (PID) controllers, process controllers, or other types of devices that implement a control loop. The control loop may comprise feedback control loop or a feedforward control loop.

Coil 110, magnetic field sensor 120, and controller 130 communicate over various communication links. The communication links comprise metallic links, glass fibers, radio channels, or some other communication media. The links may use inter-processor communication, bus interfaces, Ethernet, WiFi, virtual switching, and/or some other communication protocol. Coil 110, magnetic field sensor 120, and controller 130 may comprise microprocessors, software, memories, transceivers, bus circuitry, and the like. The microprocessors comprise Central Processing Units (CPUs), Graphical Processing Units (GPUs), Digital Signal Processors (DSPs), Application-Specific Integrated Circuits (ASICs), Field Programmable Gate Arrays (FPGAs), analog computing circuits, and/or the like. The memories comprise Random Access Memory (RAM), flash circuitry, Hard Disk Drives (HDDs), Solid State Drives (SSDs), Non-Volatile Memory Express (NVMe) SSDs, and/or the like. The memories store software like operating systems, user applications, control applications, device applications, and the like. The microprocessors retrieve the software from the memories and execute the software to drive the operation of magnetic field detection system 100 as described herein.

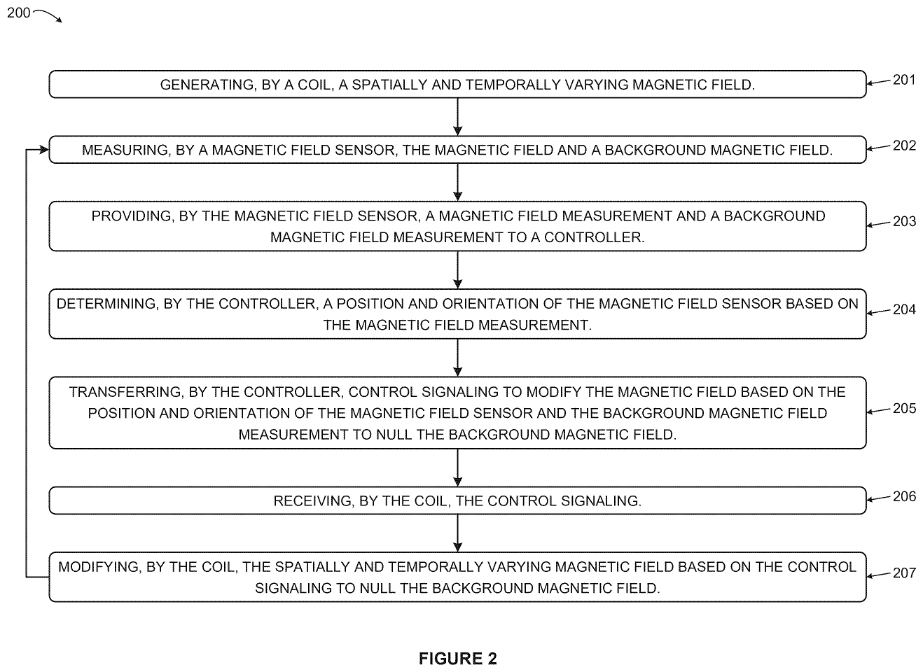

FIG. 2 illustrates process 200. Process 200 comprises an exemplary operation of magnetic field detection system 100 to mitigate background magnetic fields. Process 200 may vary in other examples. In some examples, the operations of process 200 comprise generating, by a coil (e.g., coil 110), a spatially and temporally varying magnetic field (e.g., magnetic field 111) (step 201). The operations further comprise measuring, by a magnetic field sensor (e.g., magnetic field sensor 120), the magnetic field and a background magnetic field (e.g., background magnetic field 140) (step 202). The operations further comprise providing, by the magnetic field sensor, a magnetic field measurement and a background magnetic field measurement to a controller (e.g., controller 130) (step 203). The operations further comprise determining, by the controller, a position and orientation of the magnetic field sensor based on the magnetic field measurement (step 204). The operations further comprise transferring, by the controller, control signaling to modify the magnetic field based on the position and orientation of the magnetic field sensor and the background magnetic field measurement to null the background magnetic field (step 205). The operations further comprise receiving, by the coil, the control signaling (step 206). The operations further comprise modifying, by the coil, the spatially and temporally varying magnetic field based on the control signaling to null the background magnetic field (step 207). In some examples, process 200 repeats and returns to step 202.

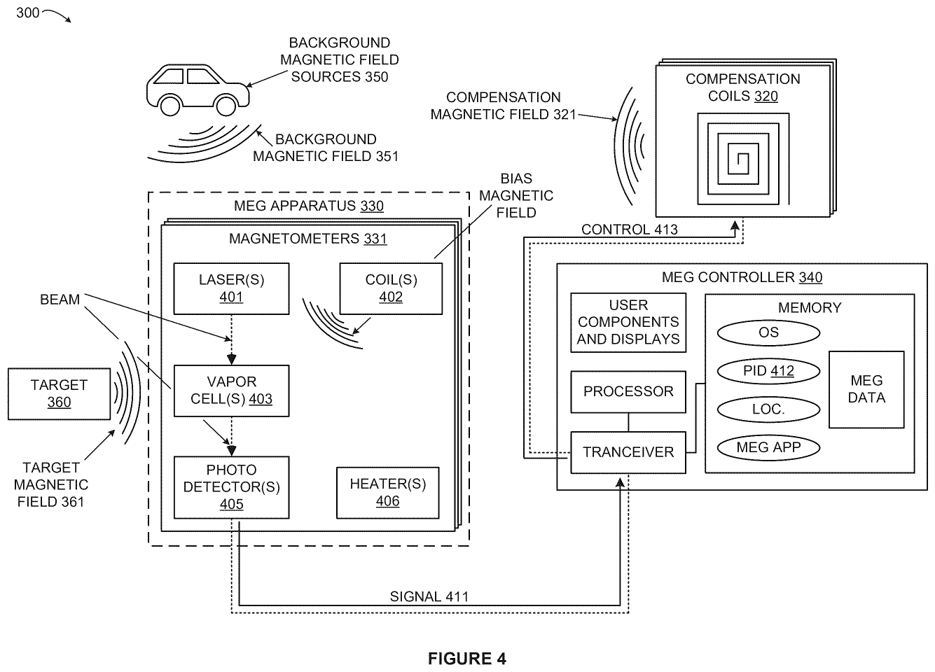

FIG. 3 illustrates an example of MEG system 300, background magnetic field sources 350, background magnetic field 351, and target 360. MEG system 300 performs operations like detecting magnetic fields, mitigating environmental magnetic noise at desired locations, and relating the detecting magnetic fields to neural activity for use in medical applications. Exemplary medical applications include identifying brain activity and diagnosing conditions like stroke, epilepsy, brain injuries, brain disorders, and/or other types of medical conditions relating to brain/neuron activity. MEG system 300 comprises an example of magnetic field detection system 100 illustrated in FIG. 1, however magnetic field detection system 100 may differ. In some examples, MEG system 300 comprises coil array 310, MEG apparatus 330, and MEG controller 340. Coil array 310 comprises two panels that mount compensation coils 320. Compensation coils 320 produce compensation magnetic field 321. The view of compensation coils 320 mounted to the right-side panel of coil array 310 are omitted from FIG. 3 for clarity. In other examples, coil array 310 may comprise additional or fewer panels than illustrated in FIG. 3. MEG apparatus 330 comprises magnetometers 331. Magnetometers 331 form a conformal MEG magnetometer array contoured to the scalp of target 360. Target 360 comprises a human head, however target 360 may comprise any magnetic field source (e.g., the human heart), including non-biological magnetic field sources. Target produces target magnetic field 361, however target magnetic field 361 (illustrated in FIG. 4) is omitted from FIG. 3 for clarity. Coil array 310, MEG apparatus 330, and target 360 are typically located in a magnetically shielded room constructed from mu-metal or another applicable material, however the magnetically shielded room is omitted for clarity. In other examples, MEG system 300 may include additional or different components than illustrated in FIG. 1.

In some examples, background magnetic field sources 350 generates background magnetic field 351. Background magnetic field sources 350 are illustrated as a mobile phone, a car, and a building, however background magnetic field sources 350 may comprise any object with the capability to produce an unwanted magnetic field. Background magnetic field 351 reaches the location of MEG apparatus 330. In examples where MEG apparatus 330 is located inside a magnetically shielded room, background magnetic field 351 (although reduced) still reaches the location of MEG apparatus 330 and may interfere in the operation of magnetometers 331. Due to the dynamic nature of background magnetic field sources 350, background magnetic field 351 is spatially and temporally varying (i.e., the strength and magnitude of background magnetic field 351 changes over time and from location-to-location).

MEG controller 340 generates and transfers control signaling to coil array 310. The control signaling may comprise electrical current directed to ones of compensation coils 320. MEG controller 340 may control the voltage, current level, amplitude, phase, frequency, modulation pattern, and/or other aspect of the current supplied to each of compensation coils 320 that drives compensation coils 320 to generate magnetic waves to form compensation magnetic field 321. Alternatively, MEG controller 340 may transfer digital or analog communication signaling to an onboard controller(s) of coil array 310 and the onboard controller may control the voltage, current level, amplitude, phase, frequency, modulation pattern, and/or other aspect of the current supplied to each of compensation coils 320 based on the signaling.

Compensation coils 320 receive their respective electrical currents and each generate magnetic waves that form compensation magnetic field 321. Compensation magnetic field 321 is a spatially and temporally varying magnetic field. The location, strength, direction, shape, and/or other characteristics of compensation magnetic field 321 depend in part on the characteristics of the electrical current supplied to compensation coils 320. For example, MEG controller 340 may select the voltage, current level, amplitude, phase, frequency, and modulation pattern of the supplied current to control the location, strength, direction, shape, and/or other characteristics of compensation magnetic field 321.

Target 360 generates target magnetic field 361. Target magnetic field 361 is created by neural activity in the brain of target 360. The neural activity comprises intercellular electromagnetic signals. The magnetic component of these electromagnetic signals forms target magnetic field 361. Target magnetic field 361 emanates from the head of target 360 and may be measured by magnetometers 331. Similar to compensation magnetic field 321 and background magnetic field 351, target magnetic field 361 may spatially and temporally vary.

MEG apparatus 330 is placed on the head of target 360. MEG apparatus is an example of a conformal MEG device. In conformal MEG, magnetometers are contoured to the shape of the wearer's head to increase the efficacy of the magnetometer array. An operator adjusts the locations/orientations of magnetometers 331 to conform magnetometers 331 to the shape of target 360's head. Once conformed, MEG controller 340 activates magnetometers 331. MEG controller 340 typically implements a sensor localization process to determine the relative locations and orientations of magnetometers 331 with respect to each other. MEG controller 340 then plots the relative locations/orientations of magnetometers 331 in a shared coordinate system. MEG controller 340 may localize magnetometers 331 using any localization process like optical scanning, physical measuring, physical constraining, magnetic field localization, and the like. For example, MEG apparatus 330 may comprise onboard coils the produce localization fields and sensor slots that physically constrain magnetometers 331 and MEG controller 340 may determine the relative locations/orientations of magnetometers 331 based on the measured strengths of the localization fields and the physical constraints placed on magnetometers 331.

Once localized, MEG controller 340 controls magnetometers 331 to begin measuring magnetic fields. Magnetometers 331 the strength and direction of compensation magnetic field 321, background magnetic field 351, and target magnetic field 361. Magnetometers 331 report the magnetic field measurements to MEG controller 340. At this point, background magnetic field 351 is not compensated (i.e., MEG controller 340 has not adjusted compensation magnetic field 321 to null background magnetic field 351). Additionally, target 360 may move their head or walk around the magnetically shielded room changing the absolute locations and orientations of magnetometers 331. It should be appreciated that the relative locations/orientations of magnetometers 331 determined during sensor localization remain unchanged.

To compensate for background magnetic field 351, MEG controller 340 begins determining the locations and orientations of magnetometers 331. MEG controller 340 may determine the locations continuously or periodically as the locations/orientations change over time from the movement of target 360. MEG controller 340 adjusts the control signaling to coil array 310 to drive compensation coils 320 to generate three homogenous sinusoidal magnetic fields and three gradient sinusoidal magnetic fields that form compensation magnetic field 321. A sinusoidal magnetic field is a magnetic field with a strength that varies over time in a sinusoidal pattern. The strength of the homogenous fields is uniform in the vicinity of MEG apparatus 330 but the directions are of the homogenous fields are different (e.g., aligned along an x, y, and z-axis). The strength of the three gradient magnetic fields varies in the vicinity of MEG apparatus 330 and the direction of the gradient fields are different. In other examples, MEG controller 340 may drive compensation coils 320 to produce additional gradient and/or homogenous magnetic fields.

Magnetometers 331 measure the strength and direction of the three sinusoidal homogenous and three sinusoidal gradient fields of compensation magnetic field 321 and reports the measurements to MEG controller 340. Magnetometer 331 makes additional measurements of the strength and direction of target magnetic field 361 and background magnetic field 351. Magnetometers 331 report these additional measurements to MEG controller 340. MEG controller 340 determines the orientations of the magnetometers based on the measurements of the homogenous sinusoidal magnetic fields. In this example, magnetometers 331 are directional (i.e., the magnitude of a sensed magnetic field increases as the field's direction aligns with sensing axis of the magnetometer). Since the direction and strength of the homogenous magnetic fields are known, MEG controller 340 correlates the reported strength of the homogenous magnetic fields to the orientation of each of magnetometers 331. For example, MEG controller 340 may drive compensation coils 320 to generate a homogenous magnetic field oriented along a vector at a strength of B towards one of magnetometers 331. The sensing axis of the magnetometer may be oriented at some angle θ with respect to the direction of magnetic field vector (e.g., θ=0° indicates the sensing axis is parallel with the vector, θ=180° indicates the sensing axis is anti-parallel with the vector, and θ=90° indicates the sensing axis is perpendicular with the vector). The directional magnetometer may measure and report the strength of the field as B_{sensed}. MEG controller 340 may then apply the following equation to determine the orientation of the magnetometer:

$$θ = {cos}^{- 1} \frac{{B}_{sensed}}{\vert B \vert}$$

where θ is the angle between the direction of the field and the magnetometers sensing axis, B_{sensed} is the measured field strength, and B is the actual field strength. MEG controller 340 may repeat this process along additional vectors (e.g., the x, y, and z-axis) to determine the orientation of the magnetometer.

MEG controller 340 determines the locations of magnetometers 331 based on the measurements of the gradient sinusoidal magnetic fields. As stated above, the strength of the gradient sinusoidal magnetic fields changes as a function of the distance from compensation coils 320. Since the direction and strength of the gradient magnetic fields are known, MEG controller 340 correlates the reported strength of the gradient magnetic fields to the locations of magnetometers 331. For example, MEG controller 340 may host a data structure that relates the distance from coil array 310 to an expected field strength along an x, y, and z-axis. MEG controller 340 may compare reported field strengths reported by magnetometers 331 to the expected field strengths, determine the distances for magnetometers 331 along the x, y, and z-axis based on the comparison, and determine spatial locations for magnetometers 331 based on the distances.

Once magnetometers 331 are located, MEG controller 340 generates additional control signaling to modify compensation magnetic field 321 to null background magnetic field 351. A magnetic field may be nulled by generating a magnetic field of equal magnitude but opposite direction (i.e., similar to the concept of destructive interference) in the same location as the field to be nulled. MEG controller 340 determines the direction and strength of background magnetic field 351 based on the measurements reported by magnetometers 331. MEG controller 340 modifies the voltage, current level, amplitude, phase, and/or frequency of the current supplied to compensation coils 320 to adjust compensation magnetic field 321 based on the absolute locations/orientations of magnetometers 331 and the strength/direction of background magnetic field 351. The adjustment tunes compensation magnetic field 321 to be the same magnitude but directionally opposed to background magnetic field 351 at the locations of magnetometers 331. The resulting interaction between compensation magnetic field 321 and background magnetic field 351 creates a volume around magnetometers 331 that is effectively free of magnetic field noise.

Magnetometers 331 continue to measure and report the strength and direction of compensation magnetic field 321, background magnetic field 351, and target magnetic field 361 to MEG controller 340. MEG controller 440 performs a source localization process based on the reported measurements of target magnetic field 361 and the relative locations/orientations of magnetometers 331 determined during the sensor localization process to generate a MEG image. Source localization entails relating the locations/orientations of magnetometers 331 and strength/direction of target magnetic field 361 to the location of the neural activity in the brain of target 360. The MEG image depicts the brain activity of target 360 in Three-Dimensions (3D). As target 360 continues to move and background magnetic field 351 continues to change, MEG controller 340 continues determining the locations/orientations of magnetometers 331 and adjusting compensation magnetic field 321 to null background magnetic field 351 as described above. The above-described operations may be performed simultaneously or in a time-multiplexed way.

Although the above examples are discussed with relation to MEG, other MXG systems for imaging electromagnetic biological signals are contemplated herein. For example, MEG system 300 may instead or additionally comprise an Electroencephalography (EEG) system, a Magnetocardiography (MCG) system, a Magnetogastrography (MGG) system, a Magnetomyography (MMG) system, Magnetoneurography (MNG), and/or another type of anatomical magnetic or electric sensing technology. It should be appreciated that the shape of MEG apparatus 330 would change when the magnetic imaging modality changes. For example, if MEG apparatus 330 instead comprises a hybrid EEG/MEG system, MEG apparatus 330 may comprise an EEG/MEG apparatus and be shaped to fit and conform magnetometers 331 and EEG electrodes to target 360's head. For example, if MEG apparatus 330 instead comprises an MCG system, MEG apparatus 330 may comprise an MCG apparatus and be shaped to fit and conform magnetometers 331 to target 360's chest. For example, if MEG apparatus 330 instead comprises an MGG system, MEG apparatus 330 may comprise a MGG apparatus and be shaped to fit and conform magnetometers 331 to target 360's abdomen. For example, if MEG apparatus 330 instead comprises an MMG system, MEG apparatus 330 may comprise a MMG apparatus and be shaped to fit and conform magnetometers 331 to target 360's arm or leg. For example, if MEG apparatus 330 instead comprises an MNG system, MEG apparatus 330 may comprise a MNG apparatus and be shaped to fit and conform magnetometers 331 to target 360's back. It should be appreciated that in all of the above described MXG systems, the controller adjusts a compensation magnetic field based on the field measurements reported by magnetometer array conformed to target 360 and the location/orientation of the magnetometer array to null the background magnetic field at the location/orientation of the MXG apparatus.

FIG. 4 illustrates examples of compensation coils 320, magnetometers 331, MEG controller 340 in MEG system 300. Compensation coils 320 comprise examples of coil 110 illustrated in FIG. 1, however coil 110 may differ. Magnetometers 331 comprise examples of magnetic field sensor 120 illustrated in FIG. 1, however magnetic field sensor 120 may differ. MEG controller 340 comprises an example of controller 130 illustrated in FIG. 1, however controller 130 may differ. In some examples, magnetometers 331 comprise lasers 401, coils 402, vapor cells 403, photodetectors 404, and heaters 405. In this example, the components of magnetometers 331 are referred to in the singular for sake of clarity. MEG controller 340 comprises a processor, a transceiver, memory, and user components and displays connected over bus circuitry. The memory in MEG controller 340 stores an operating system, Proportional Integral Derivative (PID) controller 412, a localization application, a MEG application, and MEG data. In other examples, compensation coils 320, magnetometers 331, MEG controller 340.

In some examples, MEG apparatus 330 comprises a rigid helmet, flexible cap, or other type of MEG headgear that mounts magnetometers 331 and is worn by target 360. MEG apparatus 330 comprises slots and mounts to conform magnetometers 331 to target 360. Magnetometers 331 typically comprise signal processors and other electronics, but they are omitted for sake of clarity. The processor in MEG controller 340 comprises a CPU, GPU, DSP, FPGA, ASIC, and/or some other type of processing circuitry. The memory comprises RAM, HDD, SSD, NVMe SSD, and the like. The processor retrieves the software from the memory and executes the software to drive the operation of the MEG system 300 as described herein. The processor may write and read the MEG data to and from the memory. The MEG data includes information like magnetometers Identifiers (IDs), magnetic field strengths, magnetic field directions, configuration parameters, performance characteristics, MEG images, and the like.

MEG apparatus 330 is worn by target 360 and conforms magnetometers 331 to target's 360 head. For example, magnetometers may move through the slots of MEG apparatus 330 to contact the surface of target 360's head and the mounts may lock the position and orientation of magnetometers 331 when in contact with target 360's head. The processor in MEG controller 340 retrieves and executes the software stored on the memory and drives compensation coils 320 to generate compensation magnetic field 321 and drives magnetometers 331 to sense magnetic fields. Compensation coils 320 and magnetometers 331 operate in response to the direction from MEG controller 340. In each magnetometer, vapor cell 403 is positioned in compensation magnetic field 321, background magnetic field 351, and target magnetic field 361. Vapor cell 403 may contain a metallic vapor like an alkali metal (e.g., rubidium), an alkali azide mixture (e.g., rubidium azide), and the like. Vapor cell 403 may also or alternatively contain a gas like an inert buffer gas (e.g., nitrogen). Heaters 405 heat vapor cell 403 to vaporize the contained material and pressurize vapor cell 403. Coil 402 generates a bias magnetic field to orient the sensing direction of the magnetometer. Coil 402 may apply frequency tones to dynamically measure the roll-off and compensate for it when the field modulation is applied outside frequency band of interest.

Laser 401 emits a pump beam that is circularly polarized at a resonant frequency of the vapor contained by vapor cell 403 to polarize the atoms. Laser 401 emits a probe beam that is linearly polarized at a non-resonant frequency of the vapor to probe the atoms. In some examples, magnetometers 331 may include multiple lasers (e.g., a pump laser and a probe laser). The probe beam enters the vapor cells where quantum interactions with the atoms in the presence of the magnetic fields alter the energy/frequency of probe beam by amounts that correlate to the field strength and direction of the magnetic fields. Photodetector 404 detects the probe beam after these alterations by the vapor atoms responsive to the magnetic field. Photodetector 404 generates signal 411 that characterizes the measurements. In some examples, a signal processor (not shown) may filter, amplify, digitize, or perform other tasks on the analog electronic signals. Photodetector 404 transfers signal 411 that carries the measurements to MEG controller 340.

The processor in MEG controller 340 processes signal 411 to generate data that characterizes the measured field strength/direction of compensation magnetic field 321, background magnetic field 351, and target magnetic field 351. The processor of MEG controller 340 retrieves and executes the location application from memory to perform sensor localization (i.e., plot the relative locations of the magnetometers in a shared coordinate system) and to determine the locations/orientations of magnetometers 331 with respect to coil array 310 based on homogenous and gradient components of compensation magnetic field 321 as described with respect to FIG. 3. The processor of MEG controller 340 retrieves and executes PID controller 412 to generate control output 413 to null background magnetic field 351 based on the locations/orientations of magnetometers 331 and the strength/direction of background magnetic field 351 as described with respect to FIG. 3. Control output 413 may comprise changes to the volage, current level, amplitude, phase, frequency, and/or modulation pattern of the current supplied to compensation coils 320. Compensation coils 320 modify compensation magnetic field 321 based on control output 413 to null background magnetic field 351. The processor of MEG controller 340 executes the MEG application to perform source localization and generate a MEG image based on the measurements of target magnetic field 361 as described with respect to FIG. 3. Compensation coils 320, magnetometers 331, and MEG controller 340 repeat the above-described process to continually null background magnetic field 351 at the locations/orientations of magnetometers 331 and to measure target magnetic field 361.

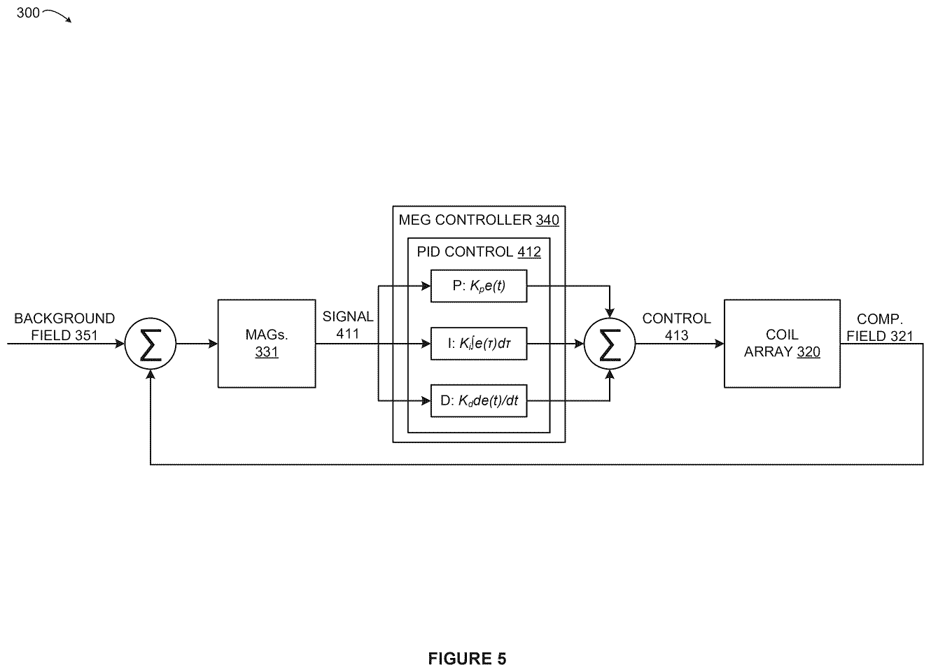

FIG. 5 illustrates an example operation of MEG system 300. MEG system 300 may implement the PID control loop illustrated in FIG. 5. In other examples, MEG system 300 may implement a different feedback/feedforward control scheme like lead-lag compensation, state feedback control, notch and resonant control, linear quadratic regulator control, model predictive control, and the like. In some examples, magnetometers 331 initially measure compensation field magnetic 321 and background magnetic field 351 to generate signal 411. Signal 411 characterizes the strength and/or direction of the sensed fields. Singal 411 is representative of the error term in the PID control scheme. Magnetometers 331 provide signal to MEG controller 340. MEG controller 340 determines the locations/orientations of magnetometers 331 and provides signal 411 and the location/orientation of PID controller 412.

PID controller 412 comprises PID functions to zero background magnetic field 351 based on signal 411 and the locations/orientations of magnetometers 331. As illustrated in FIG. 5, the PID functions comprise:

$$\begin{matrix}P - {K}_{p} e ( t ) \\ I = {K}_{i} \int e ( τ ) d τ \\ D = {K}_{d} \frac{de ( t )}{dt}\end{matrix}$$

where P, I, and D are the proportional, integral, and derivative terms, Kₚ, Kᵢ, and K_{d} are the PID coefficients, and e(τ) and e(τ) are the error values. The PID controller calculates a proportional term (P), integral term (I), and derivative term (D) based on the signal 411 and the location/orientation determined by MEG controller 340 and sums the terms to generate a control output 413. Control output 413 typically adjusts the current supplied to compensation coils 320 to align the magnitude of compensation magnetic field 321 with the magnitude of background magnetic field 351 and to align the direction of compensation magnetic field 321 to be anti-parallel with the direction of background magnetic field 351. MEG controller 340 supplies control output 413 to compensation coils 320 which adjusts compensation magnetic field 321 accordingly. MEG system 300 repeats the PID control loop illustrated in FIG. 5 to account for changes to background magnetic field 351 and the locations/orientations of magnetometers 331.

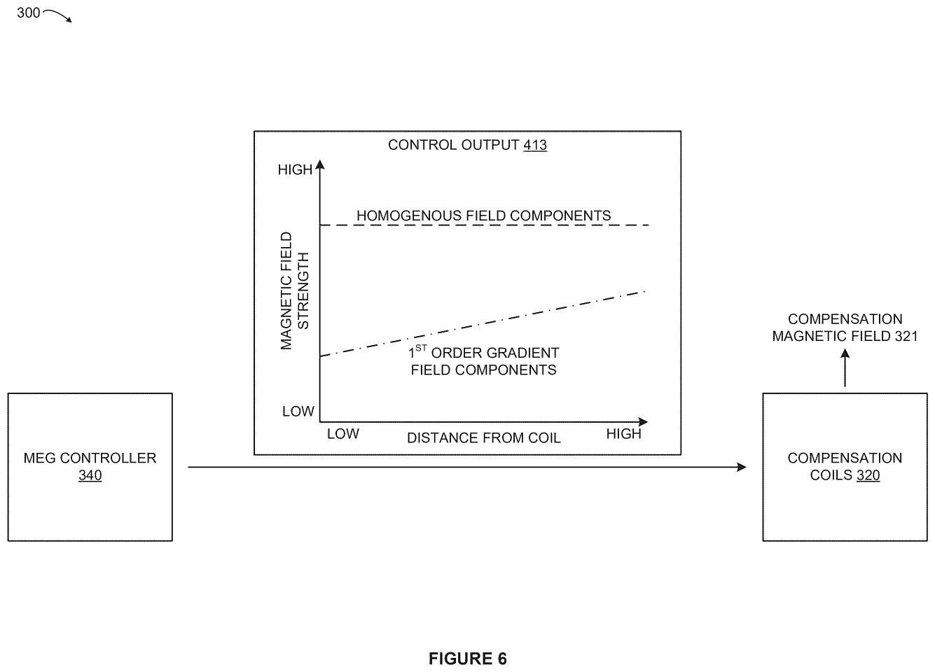

FIG. 6 illustrates an example operation of MEG system 300. MEG controller 340 provides control output 413 to compensation coils 320. Control output 413 drive compensation coils 320 to produce compensation magnetic field 321 with the spatial field components depicted in the chart illustrated in FIG. 6. The x-axis of the chart represents the distance from coil in an exemplary range LOW to HIGH. The y-axis of the chart represents magnetic field strength in an exemplary range LOW to HIGH. The ranges are exemplary and may comprise numeric values in other examples. The magnetic field components illustrated in FIG. 3 may be used to determine the locations/orientations of magnetometers 331 with respect to compensation coils 320 and to compensate background magnetic field 351. Alternatively, control output 413 may include additional field components to compensate background magnetic field 351. As illustrated in the chart, the field strength of the homogenous field components is constant with respect to distance while the field strength of the 1^{st} order gradient field components changes as a function of distance. Compensation coils 320 produces compensation magnetic field 321 based on control output 413. Compensation coils 320 typically produce three or more homogeneous sinusoidal magnetic fields to determine the orientations magnetometers 331. MEG controller 340 may determine the orientations by detecting the amplitudes of the modulation signals. Compensation coils 320 typically produces three or more sinusoidal first-order magnetic field gradients to determine the positions magnetometers 331.

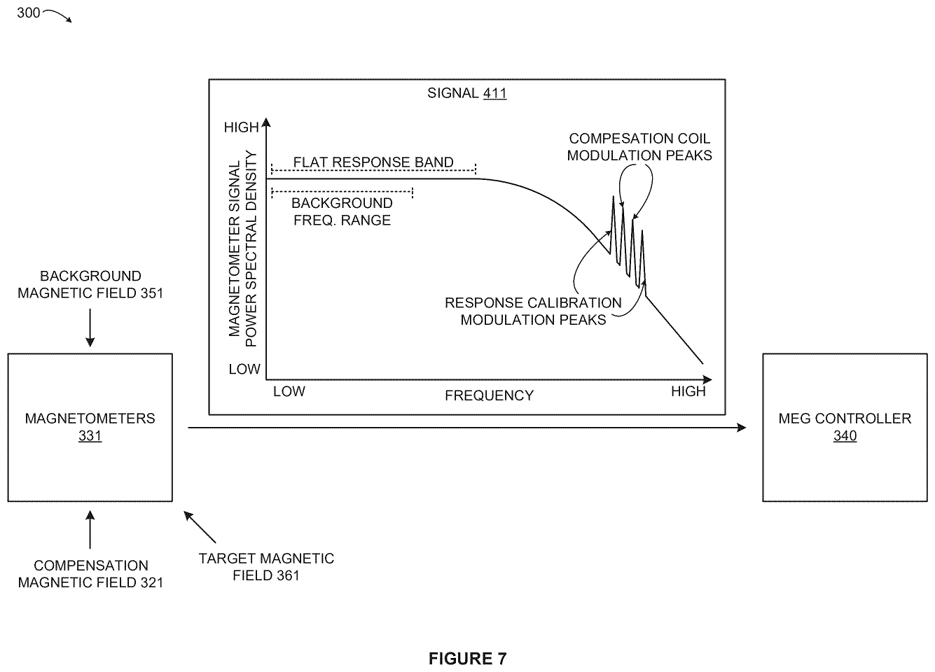

FIG. 7 illustrates an example operation of MEG system 300. In some example, magnetometers 331 sense compensation magnetic field 321, background magnetic field 351, and target magnetic field 361. Magnetometers generate signal 411 based on the sensing and transfer signal 411 to MEG controller 340. Signal 411 includes information that represents the chart illustrated in FIG. 7. The chart depicts a spectrum of the magnetic fields applied by compensation coils 320 and detected by magnetometers 331. The x-axis of the chart represents frequency in the exemplary range LOW to HIGH and the y-axis of the chart represents magnetometer spectral power spectral density in the exemplary range LOW to HIGH. In other examples, numeric values may be used.

Magnetometers 331 have a limited sensing bandwidth (sometimes referred to as the flat response band). Magnetometers 331 have a flat response band at lower frequencies, and the response band decreases at higher frequencies (i.e., as the frequency of a magnetic field increases, the ability of magnetometers 331 to measure the magnetic field decreases passed the flat response band. As depicted by the chart, the frequency range of background magnetic field 351 falls within the flat response band of magnetometers 331. The frequency range of target magnetic field 360 is generally between of 1-100 Hz and may fall within or extend past the flat response band of magnetometers 331.

If the flat response band of magnetometers 331 is larger than the frequency band of target magnetic field 360 (e.g., the frequency of target magnetic field 360 is with the bandwidth of magnetometers 331), field modulation within the flat response band is applied by compensation coils 320 to determine the locations of magnetometers 331. For example, MEG controller 340 may drive compensation coils 320 to generate spatially and temporally varying magnetic fields at a first frequency and generate magnetic field gradients at a second frequency to form compensation magnetic field 321. The first and second frequency may fall with the flat response band of magnetometers 321 and may be the same or different. MEG controller 340 may then determine the positions/orientations of magnetometers 331 based on the magnetic field measurement of the spatially and temporally varying magnetic fields generated at the first frequency and the magnetic field gradients generated at the second frequency.

Alternatively, if the flat response band of magnetometers 331 is smaller than the frequency band of target magnetic field 360 (e.g., at least a portion of target magnetic field 360 is outside the bandwidth of magnetometers 331), field modulation outside the flat response band can be applied by compensation coils 320 (depicted as compensation coil modulation peaks in the chart illustrated in FIG. 7) to determine the locations of magnetometers 331. In order to calibrate the response shape of magnetometers 331 dynamically, additional tones (depicted as response calibration modulation peaks in the chart illustrated in FIG. 7) can be applied by coils 402 in magnetometers 331. For example, MEG controller 340 may drive compensation coils 320 to generate spatially and temporally varying magnetic fields at a first frequency and generate magnetic field gradients at a second frequency to form compensation magnetic field 321. These frequencies may fall outside of the flat response band of magnetometers 331 and may be equal or different. MEG controller 340 may drive magnetometers 331 to apply additional frequency tones near (e.g., within 5 Hz) a frequency of interest in the response curve of magnetometers 331. MEG controller 340 may infer the response of magnetometers 331 outside the flat response band based on the application of the additional frequency tone near the frequency of interest in the response curve to extract information about the position and orientation of the magnetometers 331.

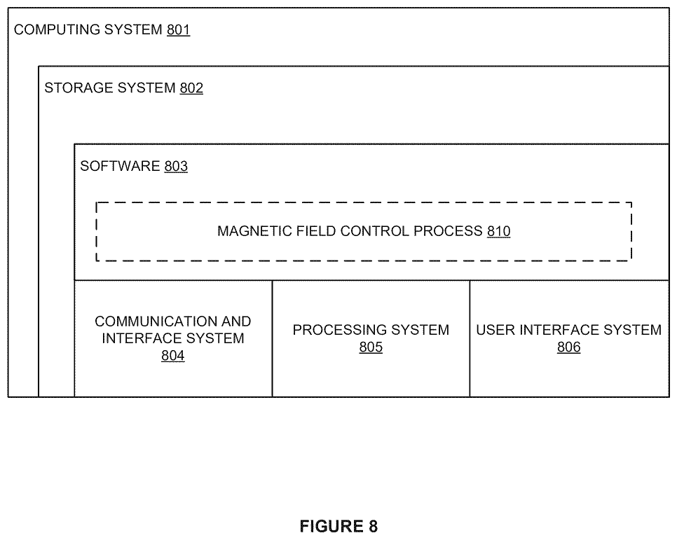

FIG. 8 illustrates computing system 801. Computing system 801 is representative of any system or collection of systems with which the various operational architectures, processes, scenarios, and sequences disclosed herein for performing dynamic global magnetic field control for mobile magnetic field measurement. For example, computing system 801 may be representative of controller 130, MEG controller 340, processing circuitry embedded in magnetic field sensor 120, processing circuitry embedded in magnetometers 331, processing circuitry embedded in coil array 310, processing circuitry embedded or operatively coupled with coil 110, and/or any other computing device contemplated herein. Computing system 801 may be implemented as a single apparatus, system, or device or may be implemented in a distributed manner as multiple apparatuses, systems, or devices. Computing system 801 includes, but is not limited to, storage system 802, software 803, communication interface system 804, processing system 805, and user interface system 806. Processing system 805 is operatively coupled with storage system 802, communication interface system 804, and user interface system 806.

Processing system 805 loads and executes software 803 from storage system 802. Software 803 includes and implements magnetic field control process 810, which is representative of any of the magnetic field control processes described with respect to the preceding Figures, including but not limited to the control operations for detecting spatially and temporally varying magnetic fields, determining the location and orientation of a magnetic field sensor, and compensating the magnetic fields at the location of a magnetic field sensor based on the measured background fields and described with respect to the preceding Figures. For example, magnetic field control process 810 may be representative of process 200 illustrated in FIG. 2 and/or any other magnetic field detection control process described herein. When executed by processing system 805 to perform dynamic global magnetic field control for mobile magnetic field measurement, software 803 directs processing system 805 to operate as described herein for at least the various processes, operational scenarios, and sequences discussed in the foregoing implementations. Computing system 801 may optionally include additional devices, features, or functionality not discussed for purposes of brevity.

Processing system 805 may comprise a micro-processor and other circuitry that retrieves and executes software 803 from storage system 802. Processing system 805 may be implemented within a single processing device but may also be distributed across multiple processing devices or sub-systems that cooperate in executing program instructions. Examples of processing system 805 include general purpose CPUs, GPUs, DSPs, ASICs, FPGAs, analog computing devices, and logic devices, as well as any other type of processing device, combinations, or variations thereof.

Storage system 802 may comprise any computer readable storage media readable by processing system 805 and capable of storing software 803. Storage system 802 may include volatile, nonvolatile, removable, and/or non-removable media implemented in any method or technology for storage of information, such as computer readable instructions, data structures, program modules, or other data. Examples of storage media include RAM, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 802 may also include computer readable communication media over which at least some of software 803 may be communicated internally or externally. Storage system 802 may be implemented as a single storage device but may also be implemented across multiple storage devices or sub-systems co-located or distributed relative to each other. Storage system 802 may comprise additional elements, such as a controller, capable of communicating with processing system 805 or possibly other systems.

Software 803 (including magnetic field control process 810) may be implemented in program instructions and among other functions may, when executed by processing system 805, direct processing system 805 to operate as described with respect to the various operational scenarios, sequences, and processes illustrated herein. For example, software 803 may include program instructions for measuring field components of a spatially and temporally varying magnetic field and controlling a compensation coil array based on the measurements.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 803 may include additional processes, programs, or components, such as operating system software, virtualization software, or other application software. Software 803 may also comprise firmware or some other form of machine-readable processing instructions executable by processing system 805.

In general, software 803 may, when loaded into processing system 805 and executed, transform a suitable apparatus, system, or device (of which computing system 801 is representative) overall from a general-purpose computing system into a special-purpose computing system customized to perform dynamic global magnetic field control for mobile magnetic field measurement as described herein. Indeed, encoding software 803 on storage system 802 may transform the physical structure of storage system 802. The specific transformation of the physical structure may depend on various factors in different implementations of this description. Examples of such factors may include, but are not limited to, the technology used to implement the storage media of storage system 802 and whether the computer-storage media are characterized as primary or secondary storage, as well as other factors.

For example, if the computer readable storage media are implemented as semiconductor-based memory, software 803 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 804 may include communication connections and devices that allow for communication with other computing systems (not shown) over communication networks (not shown). Examples of connections and devices that together allow for inter-system communication may include network interface cards, antennas, power amplifiers, radiofrequency circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 801 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of networks, or variation thereof. The aforementioned communication networks and protocols are well known and an extended discussion of them is omitted for the sake of brevity.

While some examples provided herein are described in the context of computing devices for controlling magnetic fields, it should be understood that the control systems and methods described herein are not limited to such embodiments and may apply to a variety of other environments and their associated systems. As will be appreciated by one skilled in the art, aspects of the present invention may be embodied as a system, method, computer program product, and other configurable systems. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

These and other changes can be made to the technology in light of the above Technical Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Technical Description section explicitly defines such terms. Accordingly, the actual scope of the technology encompasses not only the disclosed examples, but also all equivalent ways of practicing or implementing the technology under the claims.

What is claimed is:

1. A system comprising:
a coil, a magnetic field sensor, and a controller;
the coil configured to generate a spatially and temporally varying magnetic field;
the magnetic field sensor configured to:
measure the magnetic field and a background magnetic field; and
provide a magnetic field measurement and a background magnetic field measurement to the controller;
the controller configured to:
determine a position and orientation of the magnetic field sensor based on the magnetic field measurement; and
transfer control signaling to modify the magnetic field based on the position and orientation of the magnetic field sensor and the background magnetic field measurement to null the background magnetic field; and
the coil further configured to:
receive the control signaling; and
modify the spatially and temporally varying magnetic field based on the control signaling to null the background magnetic field.

2. The system of claim 1 further comprising a Magnetoencephalography (MEG) apparatus configured to mount the magnetic field sensor.

3. The system of claim 1 further comprising a Magnetocardiography (MCG) apparatus configured to mount the magnetic field sensor.

4. The system of claim 1 further comprising a Magnetoneurography (MNG) apparatus configured to mount the magnetic field sensor.

5. The system of claim 1 further comprising a Magnetomyography (MMG) apparatus configured to mount the magnetic field sensor.

6. The system of claim 1 further comprising a Magnetogastrography (MGG) apparatus configured to mount the magnetic field sensor.

7. The system of claim 1 wherein the magnetic field sensor comprises a magnetometer.

8. The system of claim 1 wherein the magnetic field sensor comprises an atomic magnetometer.

9. The system of claim 1 wherein the magnetic field sensor comprises an Optically Pumped Magnetometer (OPM).

10. The system of claim 1 wherein the magnetic field sensor comprises a Nitrogen-Vacancy Center (NV).

11. The system of claim 1 wherein the magnetic field sensor comprises a Magnetoresistive (MR) sensor.

12. The system of claim 1 wherein the coil comprises one coil of a coil array and the coil array is configured to:
generate the spatially and temporally varying magnetic field;
receive the control signaling, and
modify the spatially and temporally varying magnetic field based on the control signaling to null the background magnetic field.

13. The system of claim 12 wherein:
the coil array is configured to generate a homogenous magnetic field at a first frequency and generate a magnetic field gradient at a second frequency to generate the spatially and temporally varying magnetic field; and
the controller is configured to determine the position and orientation of the magnetic field sensor based on the magnetic field measurement of the homogenous magnetic field generated at the first frequency and the magnetic field gradient generated at the second frequency.

14. The system of claim 13 wherein the first frequency and the second frequency are within a flat response band of a response curve of the magnetic field sensor.

15. The apparatus of claim 13 wherein the first frequency and the second frequency are outside a flat response band of a response curve of the magnetic field sensor; and
the controller is configured to infer a response of the magnetic field sensor through measurement of the response curve outside the flat response band.

16. The apparatus of claim 15 wherein the controller is further configured to:
drive the magnetic field sensor to apply an additional frequency tone near a frequency of interest in the response curve; and
infer the response of the magnetic field sensor outside the flat response band based on the application of the additional frequency tone near the frequency of interest in the response curve to extract information about the position and orientation of the magnetic field sensor.

17. A method comprising:
generating, by a coil, a spatially and temporally varying magnetic field;
measuring, by a magnetic field sensor, the magnetic field and a background magnetic field;
providing, by a magnetic field sensor, a magnetic field measurement and a background magnetic field measurement to a controller;
determining, by the controller, a position and orientation of the magnetic field sensor based on the magnetic field measurement;
transferring, by the controller, control signaling to modify the magnetic field based on the position and orientation of the magnetic field sensor and the background magnetic field measurement to null the background magnetic field; and
receiving, by the coil, the control signaling; and
modifying, by the coil, the spatially and temporally varying magnetic field based on the control signaling to null the background magnetic field.

18. The method of claim 17 wherein:
the coil comprises one coil of a coil array;
generating, by the coil, the spatially and temporally varying magnetic field comprises generating, by the coil array, the spatially and temporally varying magnetic field;
receiving, by the coil, the control signaling comprises receiving, by the coil array, the control signaling; and
modifying, by the coil, the spatially and temporally varying magnetic field based on the control signaling to null the background magnetic field comprises modifying, by the coil array, the spatially and temporally varying magnetic field based on the control signaling to null the background magnetic field.

19. The method of claim 18 wherein:
generating, by the coil array, the spatially and temporally varying magnetic field comprises generating, by the coil array, a homogenous magnetic field at a first frequency and generating a magnetic field gradient at a second frequency; and
determining, by the controller, the position and orientation of the magnetic field sensor comprises determining, by the controller, the position and orientation of the magnetic field sensor based on the homogenous magnetic field generated at the first frequency and the magnetic field gradient generated at the second frequency.

20. The method of claim 19 wherein the first frequency and the second frequency are within a flat response band of a response curve of the magnetic field sensor.

21. The method of claim 19 wherein the first frequency and the second frequency are outside a flat response band of a response curve of the magnetic field sensor; and further comprising:
driving, by the controller, the magnetic field sensor to apply an additional frequency tone near a frequency of interest in the response curve; and
inferring, by the controller, a response of the magnetic field sensor through measurement of the response curve outside the flat response band based on the application of the additional frequency tone near the frequency of interest in the response curve to extract information about the position and orientation of the magnetic field sensor.

22. One or more non-transitory computer readable storage media having program instructions stored thereon, wherein the program instructions, when executed by a computing system, direct the computing system to perform operations, the operations comprising:
controlling a coil to generate a spatially and temporally varying magnetic field;
directing a magnetic field sensor to measure the magnetic field and a background magnetic field;
obtaining a magnetic field measurement and a background magnetic field measurement;
determining a location and orientation of the magnetic field sensor based on the magnetic field measurement; and
controlling the coil to modify the magnetic field based on the location and orientation of the magnetic field sensor and the background magnetic field measurement to null the background magnetic field.
